(19) United States
(12) Patent Application Publication
(10) Pub. No.: US 2003/0180936 A1
(43) Pub. Date: Sep. 25, 2003

(54) Method for the purification, production and formulation of oncolytic adenoviruses

(76) Inventors: Bahram Eric Memarzadeh, San Carlos, CA (US); Rukmini Pennathur-Das, Los Altos, CA (US); Joseph Wypych, Tracy, CA (US); De Chao Yu, Foster City, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/254,156

(22) Filed: Sep. 24, 2002

Related U.S. Application Data
(63) Continuation-in-part of application No. 10/099,513, filed on Mar. 15, 2002.

(51) Int. Cl. C12N 7/02; C12Q 1/70; C12N 13/00; C12N 7/00; C12N 7/01

(52) U.S. Cl. 435/239; 435/5; 435/235.1; 435/173.9

(57) ABSTRACT

A process is provided for the production of substantially pure replication competent adenovirus, together with improved formulations for maintenance of infectivity following storage. In the process, virus infected cells are lysed with detergent. An initial purification step utilizes a pass through a high throughput ion exchange filter. The eluant is treated with nuclease, then refiltered on a high throughput ion exchange filter. The virus formulation provides for enhanced stability of liquid viral preparations at 5° C.

### TECHNICAL FIELD

The technical field of the invention is methods of producing, purifying and formulating replication competent adenoviral vectors.

### BACKGROUND OF THE INVENTION

The use of viruses as a cancer therapy was first explored after observations of occasional tumor regressions in cancer patients suffering from virus infections or receiving vaccinations. Although these early clinical trials were abandoned, the idea was revived after the development of genetic engineering techniques, which held the promise of enhanced efficacy and decreased toxicity. As a result of increasing knowledge of adenoviral interactions with cell cycle regulatory proteins and the experience gained from its use as a gene delivery vehicle, adenovirus has emerged as a virus that can be engineered with oncotropic properties.

Adenoviruses generally undergo an effective lytic replication cycle following infection of a host cell. In addition to lysing the infected cell, the replicative process of adenovirus blocks the transport and translation host cell mRNA, thus inhibiting protein synthesis of the infected cell. For a review of adenoviruses and adenovirus replication, see Shenk and Horwitz, Virology, third edition, Fields et al., eds., Raven Press Limited, New York (1996), Chapters 67 and 68, respectively. In addition, replication-competent adenoviruses can sensitize tumor cells to chemotherapy.

Replicative adenoviruses have been engineered to achieve selective targeting and amplification for the treatment of local and disseminated cancer. Such an agent can be delivered systemically, can be targeted to tumor cells, and can amplify its cytolytic effect in a tumor-specific manner, thereby providing substantial clinical benefit. See Henderson et al., U.S. Pat. No. 5,698,443; Hallenbeck et al., WO 96/17053. In such systems, a cell-specific transcriptional regulatory element controls the expression of a gene essential for viral replication, and thus, viral replication is limited to a cell population in which the element is functional. For example, an attenuated, replication-competent adenovirus has been generated by inserting the prostate-specific antigen (PSA) promoter and enhancer (PSE-TRE) upstream of the E1A transcription unit in adenovirus serotype 5 (Ad5), which virus demonstrates selective cytotoxicity toward PSA expressing cells in vitro and in vivo (Rodriguez et al. (1997) Cancer Res. 57:2559-2563).

Clinical studies have demonstrated the safety and feasibility of this approach, including the delivery of adenovirus to tumors through the bloodstream. Improvements in the purification and formulation of such viruses are of great interest.

Relevant Literature

U.S. Pat. No. 6,194,191 Zhang et al., discloses methods for the production and purification of adenoviral vectors. International patent application WO 99/54441 discloses methods of purifying adenovirus by means of anion exchange chromatography. International patent application WO 98/22588 discloses a method of production or purification of adenovirus particles. Another method for production of recombinant virus is disclosed in International patent application WO 98/00524, and for recombinant virus containing a therapeutic gene in International patent application WO 96/27677.

### SUMMARY OF THE INVENTION

Methods are provided for the production and purification of replication competent adenovirus, resulting in high yields and high recovery of the active adenovirus. Producer cells are cultured at a large scale in media at a low perfusion rate, then infected with replication competent adenovirus, maintained in culture for a period of time sufficient to replicate the adenovirus, and lysed with a detergent. The cell lysate is then clarified by filtration, and purified using a high throughput ion exchange filter cartridge. The eluant is treated with nuclease, then refiltered on a high throughput ion exchange filter. The methods provide for a highly efficient purification process. The purified virus may be stored frozen, lyophilized, or in liquid formulation, preferably at cool temperatures. Improved formulations for these conditions are provided.

The improved formulations of the invention contain a zwitterionic compound such as glycine and provide for stability of virus preparations for at least 21 months at 5° C. Glycine is typically present at a concentration of at least about 0.1% and not more than about 5%. In one preferred embodiment, glycine is present at a concentration of 0.5% and not more than about 1.5%, most frequently at a concentration of about 1%.

### BRIEF DESCRIPTION OF THE DRAWINGS

FIG. 1 is a schematic depicting the purification process of the invention.

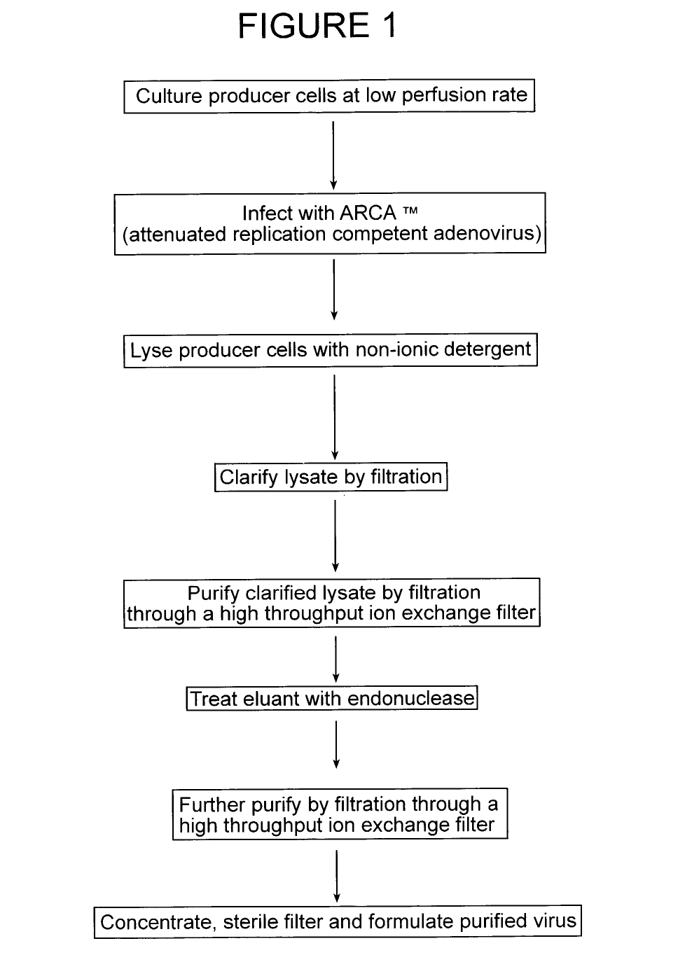

FIG. 2 is a graph depicting the differences in methods of quantitating particles in a formulation.

FIG. 3 is a graph depicting the stability of an adenoviral formulation at different temperatures.

### DETAILED DESCRIPTION OF THE EMBODIMENTS

The present invention provides a process for the production and purification of replication competent adenovirus. Cells permissive for adenovirus replication are cultured at a low perfusion rate, then infected with replication competent adenovirus. After virus replication, the cells are lysed with detergent. An initial purification step utilizes a pass through a high throughput ion exchange filter. The eluant is treated with nuclease, then refiltered on a high throughput ion exchange filter. The virus suspension is optionally sterile filtered and formulated for use. The methods of the invention provide for a substantially pure population of adenovirus, with a high yield as calculated from the original cell lysate. Usually the final yield will be at least about 80% of the adenovirus present in the lysate, more usually at least about 85% of the adenovirus present in the lysate, and preferably at least about 90% of the adenovirus present in the lysate.

An advantageous feature of the invention is the use of an ion exchange filter, for example a filter cartridge. Such filter cartridges provide for a significant increase in recovery of product when compared to the use of column resins for purification. In addition, such filter cartridges can be run with high flow rates, thereby minimizing the time required for purification, and have a high binding capacity for both adenovirus and DNA.

The invention provides the further advantage of a formulation for long-term maintenance of viral stability at a temperature (5° C.) that is practical to the therapeutic use of the virus.

The various methods and formulations of the invention will be described below. Although particular methods of purification are exemplified in the discussion below, it is understood that any of a number of alternative methods are applicable and suitable for use in practicing the invention. It will also be understood that an evaluation of the adenovirus vectors and methods of the invention may be carried out using procedures standard in the art, including the diagnostic and assessment methods described below.

The practice of the present invention will employ, unless otherwise indicated, conventional techniques of cell biology, molecular biology (including recombinant techniques), microbiology, biochemistry and immunology, which are within the scope of those of skill in the art. Such techniques are explained fully in the literature, such as, “Molecular Cloning: A Laboratory Manual”, second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Handbook of Experimental Immunology” (D. M. Weir & C. C. Blackwell, eds.); “Gene Transfer Vectors for Mammalian Cells” (J. M. Miller & M. P. Calos, eds., 1987); “Current Protocols in Molecular Biology” (F. M. Ausubel et al., eds., 1987); “PCR: The Polymerase Chain Reaction”, (Mullis et al., eds., 1994); and “Current Protocols in Immunology” (J. E. Coligan et al., eds., 1991), each of which is expressly incorporated by reference herein.

For techniques related to adenovirus, see, inter alia, Felgner and Ringold (1989) Nature 337:387-388; Berkner and Sharp (1983) Nucl. Acids Res. 11:6003-6020; Graham (1984) EMBO J. 3:2917-2922; Bett et al. (1993) J. Virology 67:5911-5921; Bett et al. (1994) Proc. Natl. Acad. Sci. USA 91:8802-8806.

### Definitions

Unless otherwise indicated, all terms used herein have the same meaning as they would to one skilled in the art and the practice of the present invention will employ, conventional techniques of microbiology and recombinant DNA technology, which are within the knowledge of those of skill of the art.

An “adenovirus vector” or “adenoviral vector” (used interchangeably) of the invention is a polynucleotide construct, which provides for a replication competent adenovirus; which in some embodiments will exhibit preferential replication in target cells and contain a tissue-specific transcriptional regulatory sequence linked to an adenoviral gene. In some embodiments, an adenoviral vector of the invention includes a therapeutic gene sequence, e.g., a cytokine gene sequence. Exemplary adenoviral vectors of the invention include, but are not limited to, DNA, DNA encapsulated in an adenovirus coat, adenoviral DNA packaged in another viral or viral-like form (such as herpes simplex, and AAV), adenoviral DNA encapsulated in liposomes, adenoviral DNA complexed with polylysine, adenoviral DNA complexed with synthetic polycationic molecules, conjugated with transferrin, or complexed with compounds such as PEG to immunologically “mask” the antigenicity and/or increase half-life, or conjugated to a nonviral protein.

For the purification methods of the present invention, an adenovirus vector encapsulated in adenovirus coat, or in another viral or viral-like form, will generally be the form that is purified, although the DNA and other forms may find use, for example, in the initial infection steps. The term “adenovirus”, or “adenovirus particle” may be used interchangeably to refer to such an encapsulated vector.

“Replication” and “propagation” are used interchangeably and refer to the ability of an adenovirus vector to reproduce or proliferate. These terms are well understood in the art. For purposes of this invention, replication involves production of adenovirus proteins and is generally directed to reproduction of adenovirus. Replication can be measured using assays standard in the art and described herein, such as a virus yield assay, burst assay or plaque assay. “Replication” and “propagation” include any activity directly or indirectly involved in the process of virus manufacture, including, but not limited to, viral gene expression; production of viral proteins, nucleic acids or other components; packaging of viral components into complete viruses; and cell lysis.

“Preferential replication” and “selective replication” may be used interchangeably and mean that an adenovirus replicates more in a target cell than in a non-target cell. The adenovirus may replicate at a significantly higher rate in target cells than in non target cells; for example, at least about 5-fold higher; at least about 10-fold higher; at least about 50-fold higher; at least about 100-fold higher; at least about 400- to 500-fold higher; at least about 1000-fold higher; or at least about 1×10⁶ higher. Where the replication competent adenovirus vector has such target cell specificity, the cell line and/or conditions for virus replication will be selected so as to permit replication.

A “host cell” includes an individual cell or cell culture which can be or has been a recipient of an adenoviral vector(s) of this invention. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo or in vitro with an adenoviral vector of this invention.

Host cells are capable of supporting replication of adenovirus. Host cells according to the present invention are derived from a mammalian cell and, preferably, from a primate cell such as human embryonic kidney cell. Although various primate cells are preferred and human or even human embryonic kidney cells are most preferred, any type of cell that is capable of supporting replication of the virus is acceptable in the practice of the invention. A preferred cell line for large-scale production of adenovirus is the human embryonic kidney cell line, 293, which expresses the adenoviral EIA and EIB gene products. For example, a helper cell line has been derived from the transformation of 293 human embryonic kidney cells by Adenovirus serotype 5, which cell lines are capable of supporting replication of defective, recombinant, adenoviral vectors. Other cell lines capable of producing appropriately targeted adenovirus include human LNCaP (prostate carcinoma), HBL-100 (breast epithelia), OVCAR-3 (ovarian carcinoma), and the like. Other cell types might include, but are not limited to Vero cells, CHO cells or any eukaryotic cells for which tissue culture techniques are established as long as the cells are adenovirus permissive. The term “adenovirus permissive” means that the adenovirus or adenoviral vector is able to complete the entire intracellular virus life cycle within the cellular environment.

A candidate cell line may be tested for its ability to support adenovirus replication by methods known in the art, e.g. by contacting a layer of uninfected cells, or cells infected with one or more helper viruses, with virus particles, followed by incubation of the cells. The formation of viral plaques, or cell free areas in the cell layer, is the result of cell lysis caused by the expression of certain viral products. Cell lysis is indicative of viral replication.

The present invention provides methods for the purification, and in particular embodiments, the substantial purification, of an adenoviral particle. The term “purified” as used herein, is intended to refer to a composition, isolatable from other components, wherein the adenoviral particle is purified to any degree relative to its naturally obtainable form. A purified adenoviral particle therefore also refers to an adenoviral component, free from the environment in which it may naturally occur. Generally, “purified” will refer to an adenoviral particle that has been subjected to fractionation to remove various other components, and which composition substantially retains its expressed biological activity. Where the term “substantially purified” is used, this designation will refer to a composition in which the virus particle forms the major component of the composition, such as constituting at least about 50%, usually at least about 75%, preferably at least about 90% or more of the biological constituents in the composition.

### Growth of Adenovirus Vector

The host cells are usually grown in perfused systems, which allow for the maintenance of a good culture environment of pH, CO₂ and O₂ while the cells are growing. Perfusion allows active metabolites to be removed, while the nutrients are being supplied. Medium suitable for cell culture is well known in the art, and any suitable medium can be utilized, e.g. RPMI, DMEM, etc. The medium may contain serum, e.g. FBS, or may be serum-free. Serum weaning adaptation of anchorage-dependent cells into serum-free suspension cultures has been used for the production of recombinant proteins and viral vaccines, and may find use in the present methods.

The host cells are infected with replication competent adenovirus by contacting the cells with virus under physiological conditions, permitting the uptake of virus. The host cell then replicates the virus, which can be harvested at least about 2 days after infection, and not more than about 7 days after infection; more usually after about 3 days and not more than about 5 days.

In certain embodiments, it may be useful to employ selection systems that preclude growth of undesirable cells. This may be accomplished by virtue of permanently transforming a cell line with a selectable marker or by transducing or infecting a cell line with a viral vector that encodes a selectable marker. In either situation, culture of the transformed/transduced cell with an appropriate drug or selective compound will result in the selective replication of those cells carrying the marker. Selective replication of cells carrying the marker means that culture of transformed/transduced cells in the presence of an appropriate type and concentration of drug or selective compound results in either preferential or exclusive replication of cells that carry the marker relative to cells that do not carry the marker. Examples of markers include, but are not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr, which confers resistance to methotrexate; gpt, which confers resistance to mycophenolic acid; neo, which confers resistance to the aminoglycoside G418; and hygro, which confers resistance to hygromycin.

The present invention will generally take advantage of bioreactor technology for production of virus. Growing cells according to the present invention in a bioreactor allows for large-scale production of biologically active cells capable of being infected by the adenoviral vectors of the present invention. By operating the system at a low perfusion rate and applying a high throughput scheme for purification, the invention provides a strategy that is easily scaleable to produce large quantities of highly purified product.

Bioreactors have been widely used for the production of biological products from both suspension and anchorage dependent animal cell cultures. Bioreactors for adenoviral vector production should have the characteristic of high volume-specific culture surface area in order to achieve high producer cell density and high virus yield.

Perfusion of fresh medium through the culture can be achieved by retaining the cells with a variety of devices, e.g. fiber disks, fine mesh spin filter, hollow fiber or flat plate membrane filters, settling tubes, etc. A simple perfusion process has an inflow of medium and an outflow of cells and products. Culture medium is fed to the reactor at a predetermined and constant rate, which maintains the dilution rate of the culture at a value less than the maximum specific growth rate of the cells. Culture fluid containing cells and cell products and byproducts is removed at the same rate. Instrumentation and controls are basically the same as found in other fermentors and include agitation, temperature, dissolved oxygen, and pH controls. More advanced probes and autoanalyzers for on-line and off-line measurements of turbidity (a function of particles present), capacitance (a function of viable cells present), glucose/lactate, carbonate/bicarbonate and carbon dioxide are available.

In one embodiment of the invention, suspension adapted cells are used, which can be grown in serum-containing or serum-free medium. The development of a perfused packed-bed reactor using a bed matrix of a non-woven fabric has provided a means for maintaining a perfusion culture at densities exceeding 10⁸ cells/ml of the bed volume (CelliGen™, New Brunswick Scientific, Edison, N.J.) This system comprises an improved reactor for culturing of both anchorage- and non-anchorage-dependent cells. The reactor is designed as a packed bed with a means to provide internal recirculation. Preferably, a fiber matrix carrier is placed in a basket within the reactor vessel. A top and bottom portion of the basket has holes, allowing the medium to flow through the basket. A specially designed impeller provides recirculation of the medium through the space occupied by the fiber matrix for assuring a uniform supply of nutrient and the removal of wastes. This simultaneously assures that a negligible amount of the total cell mass is suspended in the medium. The combination of the basket and the recirculation also provides a bubble-free flow of oxygenated medium through the fiber matrix. The fiber matrix is a non-woven fabric having a “pore” diameter of from 10 μm to 100 μm, providing for a high internal volume with pore volumes corresponding to 1 to 20 times the volumes of individual cells.

The Cellcube™ (Corning-Costar) module provides a large styrenic surface area for the immobilization and growth of substrate attached cells. It is an integrally encapsulated sterile single-use device that has a series of parallel culture plate joined to create thin sealed laminar flow spaces between adjacent plates. The Cellcube™ module has inlet and outlet ports that are diagonally opposite each other and help regulate the flow of media. Cells within the system reach a higher density of solution (cells/ml) than in traditional culture systems. One of the benefits of such a system is the ability to provide a gentle transition between various operating phases. The perfusion system negates the need for traditional wash steps that seek to remove serum components in a growth medium. A preferred system has an oxygenator, pH and pO₂ probes, and pumps for both re-circulation from the oxygenator to the module and continuous perfusion. The timing and the rates of re-circulation and perfusion is dependent on the seeding cell density, and the cell growth which is monitored by amounts of nutrients e.g. glucose and metabolites, e.g. lactate, etc. over time.

### Cell Harvest and Lysis

Although infection with replication competent adenovirus will result in lysis of the infected cells, it is preferable to lyse cells prior to complete lysis. A preferred method of lysis uses the addition of non-ionic detergent to the infected cells, at a final concentration of at least about 0.5% weight/volume, and not more than about 2.5% weight/volume, more usually at least about 1% weight/volume and not more than about 2% weight/volume. Non-anionic detergents include the Triton™ family of detergents, e.g. Triton™ X-15; Triton™ X-35; Triton™ X-45; Triton™ X-100; Triton™ X-102; Triton™ X-114; Triton™ X-165, etc. All of these heterogeneous detergents have a branched 8-carbon chain attached to an aromatic ring. This portion of the molecule contributes most of the hydrophobic nature of the detergent. Triton™ X-100 and NP-40 are very similar in structure and hydrophobicity and are interchangeable in most applications including cell lysis. Brij™ detergents are also similar in structure to Triton™ X detergents in that they have varying lengths of polyoxyethylene chains attached to a hydrophobic chain. However, unlike Triton™ X detergents, the Brij™ detergents do not have an aromatic ring and the length of the carbon chains can vary. Brij™ 58 is most similar to Triton™ X 100 in its hydrophobic/hydrophilic characteristics. The Tween™ detergents are nondenaturing, nonionic detergents; which are polyoxyethylene sorbitan esters of fatty acids. Tween™ 80 is derived from-oleic acid with a C₁₈ chain while Tween™ 20 is derived from lauric acid with a C₁₂ chain. The zwitterionic detergent, CHAPS, is a sulfobetaine derivative of cholic acid. This zwitterionic detergent is useful for membrane protein solubilization when protein activity is important. This detergent is useful over a wide range of pH (pH 2-12) and is easily removed from solution by dialysis due to high CMCs (8-10 mM). A preferred non-ionic detergent is Triton-X 100 or NP-40.

The detergent is contacted with the cells for a period of time sufficient to lyse the cells and remove additional adherent cells from the system. This period of time is usually at least about 30 minutes and not more than about 4 hours, more usually at least about 1 hour and not more than about 2 hours.

Before purification of the virus, the crude viral lysate needs to be clarified i.e., the membrane fragments need to be removed. Clarification is achieved by the use of depth filters consisting of a packed column of a non-absorbent material of certain porosity such that the bigger membrane debris is retained without the loss of any adenoviral particles. Depth filters are selected on the basis of mechanical retention of particles, absorption characteristics, pH value, surface quality, thickness and strength of the filter. Commercially available cartridges combine several types of filters, e.g. polypropylene, glass fibers, nitrocellulose, and the like. Typically such filters are graded for the size of particle that is excluded. For the purposes of the present invention, one or more depth filters are used for clarification, which filters will usually exclude up to about 0.2 □m particles, i.e. the filters will exclude cellular debris, but not the virus particles.

### Separation

Adenovirus particles are separated from the clarified cell lysate by anion exchange chromatography on a filter cartridge. An ion exchanger is a solid that has chemically bound charged groups to which ions are electrostatically bound; it can exchange these ions for ions in aqueous solution. When the charged group is positive, e.g. a quaternary amino group, it is a strongly basic anion exchanger. Common weakly basic anion exchangers are aromatic or aliphatic amino groups. The total capacity of an ion exchanger measures its ability to take up exchangeable groups per milligram of dry weight. The available capacity is the capacity under particular experimental conditions (i.e., pH, ionic strength). For example, the extent to which an ion exchanger is charged depends on the pH. Another factor is ionic strength because small ions near the charged groups compete with the sample molecule for these groups. The binding capacity of a particular filter can be determined by conventional methods, e.g. overloading the filter and determining the amount of virus that is bound.

As is generally known in the art, anion exchangers typically include a functional group attached to a matrix. Prior to the present invention, anion exchange media used in virus purification/analysis was typically in the form of a resin containing small porous beads, which bind molecules, internally by diffusion. Such resins are typically packed into a re-usable column. Exemplary resins include, but are not limited to polystyrene cross-linked with divinylbenzene beads, as found in Pharmacia Source Q, which is often used for analytical HPLC, and dextran attached to highly cross-linked spherical agarose beads, as found in Pharmacia Q-Sepharose XL. (See, e.g., International patent application WO 00/40702.)

The present invention takes advantage of a different type of matrix. Matrices for use in the present invention take the form of a filter membrane, which binds molecules on the surface (externally) by direct fluid convection. Preferred matrices come in a disposable capsule form ready for use. Such filter capsules or cartridges provide the advantages of: (1) faster flow rates; (2) higher binding capacity (e.g., 5e11 vp/mL, as compared to 5e12 vp/mL for standard resins); (3) a virus recovery of up to 90% (i.e., higher than the 70% recovery typically achieved using anion exchange resins); (4) no packing or cleaning validation required for clinical use; and (5) no resin lifetime issues or storage issues, when disposable filter cartridges are used.

High throughput ion exchange filters suitable for use in the methods of the present invention are known in the art and commercially available. Such filters comprise multiple layers of a filter membrane, and have a bound anion, as described above. Furthermore, preferred filters are in the form of a disposable cartridge.

For example, the Pall Mustang Q filter contains pendent quaternary amine groups in cross-linked polymeric coating of a modified hydrophilic, poyethersulfone filter. Various filters can be used, however it is preferable to have a filter that provides for a high flow rate, with a high viral and DNA binding capacity, usually a strongly basic ion exchanger. Desirably, cartridges of pleated filters with an anion exchanger will have a binding capacity of at least about 10¹² virus particles/ml, more preferably a binding capacity of at least about 5×10¹² virus particles/ml. Although not required for the purposes of the present invention, such filters often have a high capacity for DNA binding as well.

The adenovirus is loaded and eluted from the ion exchange filter at a suitable ionicity, which permits separation of the virus from proteins, DNA, detergent from lysis, and other biochemicals present in the lysate. Usually the lysate will be loaded at an ionicity that prevents most cellular and serum proteins from binding, but still permit binding of virus. With a quarternary amine group as the anion exchanger, for example when using a Pall Mustang Q filter cartridge, the loading ionicity will usually be at least about 25 mS/cm, more-usually at least about 30 mS/cm, preferably at least about 40 mS/cm. Where NaCl is the ion, it will be present at a concentration of at least about 250 mM, more usually at least about 300 mM, preferably at least about 400 mM and may be present at a concentration of at least about 450 or 500 mM.

The ionicity for optimal elution of virus is empirically selected as that which gives the best separation from major contaminants, at an ionicity higher than the loading ionicity, but below the elution of, for example, nucleic acids. As will be understood by those of skill in the art, this ionicity will vary somewhat depending upon the particular anion exchanger employed. While the optimal ionicity may vary, depending on minor differences in salts, properties of the specific anion exchangers, etc., it has been found for a quarternary amine, that virus elutes well at from about 450 to 750 mM NaCl, more usually at from about 500 to 600 mM NaCl, which provides for an excellent separation from bound DNA. More specifically, when using a Pall Mustang Q filter cartridge, virus elutes well at from about 600 to 700 mM NaCl, and may be eluted using from about 525 to 700 mM NaCl. In one preferred approach, virus is eluted with 600 mM NaCl, followed by washing the filter with 500 mM NaCl.

After the chromatography step, the eluant is treated with a nuclease, which reduces the concentration of nucleic acid (RNA and DNA) residue from the cell lysate. Use of nuclease at this point, rather than immediately post-harvest, minimizes the amount of nuclease required. The use of a second chromatography step technique after nuclease treatment ensures the removal of fragmented DNA and the nuclease.

Many nucleases are known in the art, including enzymes from the following enzyme classifications: 2.7.7.56; 3.1.4.1; 3.1.11.1; 3.1.11.2; 3.1.11.3; 3.1.11.4; 3.1.11.5; 3.1.11.6; 3.1.13.1; 3.1.13.2; 3.1.13.4; 3.1.14.1; 3.1.15.1; 3.1.16.1; 3.1.21.1; 3.1.21.2; 3.1.21.3; 3.1.21.4; 3.1.21.5; 3.1.21.6; 3.1.22.1; 3.1.22.2; 3.1.22.3; 3.1.22.4; 3.1.22.5; 3.1.25.1; 3.1.26.1; 3.1.26.2; 3.1.26.3; 3.1.26.4; 3.1.26.5; 3.1.26.6; 3.1.26.7; 3.1.26.8; 3.1.26.9; 3.1.26.10; 3.1.27.1; 3.1.27.2; 3.1.27.3; 3.1.27.4; 3.1.27.5; 3.1.27.6; 3.1.27.7; 3.1.27.8; 3.1.27.9; 3.1.27.10; 3.1.30.1; 3.1.30.2; 3.1.31.1; and 4.2.99.18. Preferred is one or a combination of broad specificity endonucleases, e.g. enzyme classification 3.1.27.5 (pancreatic ribonuclease) and 3.1.31.1 (micrococcal nuclease); and the like. In one embodiment of the invention, the nuclease is Benzonase™, a genetically engineered enzyme with both DNAse and RNAse activity. The ability of Benzonase™ to rapidly hydrolyze nucleic acids makes the enzyme useful for reducing cell lysate viscosity, and for reducing the nucleic acid load during purification, thus eliminating the interference and improving yield. Upon complete digestion, all free nucleic acids present in solution are-reduced to oligonucleotides 2 to 4 bases in length.

Following nuclease digestion, the virus is preferably run for a second time on an anion exchange filter; where the filter may be the same or different as the first filter. Where the filter is the same, it may be re-used from the initial separation, or a virgin filter may be used. Generally, the considerations for selection of an anion exchanger and for loading and elution, will be the same as those described above.

### Formulation

The eluant is optionally concentrated and diafiltered by conventional methods, e.g. with a hollow fiber concentrator. In a final preparation for use, the virus sample may be sterile filtered, e.g. for clinical use. A variety of filters suitable for this purpose are known in the art, e.g. nitrocellulose membrane filters; cellulose acetate membrane filters; PVDF (modified polyvinylidene fluoride) membrane filters; and the like. Preferred are PVDF membrane filters (for example Millipore Millipak filters). The yield from a run can be improved by pre-washing the filters with buffer, e.g. a pharmaceutically acceptable excipient. It has been found that yield is reduced by binding of virus to the filter, where the binding is saturated after a certain level.

Therefore, yield can be improved by loading a higher number of particles, so that the percentage loss is minimized. In one embodiment of the invention, at least about 0.5×10¹³ virus particles/cm² surface area of filter is loaded, more usually at least about 0.75×10¹³ virus particles/cm² surface area of filter is loaded; preferably at least about 1×10¹³ virus particles/cm² surface area of filter is loaded.

The sterile filtered virus suspension may be formulated for use in vitro or in vivo. Aqueous compositions comprise an effective amount of the virus, suspended in a pharmaceutically acceptable carrier or aqueous medium. Such compositions can also be referred to as inocula. The phrases “pharmaceutically or pharmacologically acceptable” refer to molecular entities and compositions that do not produce an adverse, allergic or other untoward reaction when administered to an animal, or a human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art.

Except insofar as any conventional media or agent is incompatible with the active ingredient, its use in the therapeutic compositions is contemplated. Supplementary active ingredients also can be incorporated into the compositions.

Formulations include injectable compositions either as liquid solutions or suspensions; solid forms suitable for solution in, or suspension in, liquid prior to injection may also be prepared. These preparations also may be emulsified. Atypical composition for such purpose comprises a pharmaceutically acceptable carrier. For instance, the composition may contain about 100 mg of human serum albumin per milliliter of phosphate buffered saline. Other pharmaceutically acceptable carriers include aqueous solutions, non-toxic excipients, including salts, preservatives, buffers and the like may be used. Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components in the pharmaceutical composition are adjusted according to well known parameters.

Formulations may be optimized for the desired storage conditions. In one embodiment of the invention, particularly with virus formulated for clinical use, the samples are stored in liquid form, preferably at cool temperatures, usually less than about 10° C., more usually less than about 5° C. For such conditions, a preferred medium for storage comprises 5% sucrose, 1% glycine, 1 mM MgCl₂, 10 mM Tris, and small amounts of a surfactant. One surfactant of interest is a non-ionic detergent, e.g. Tween 80, Tween 20, etc., at a concentration of from about 0.01% to about 0.1%, preferably about 0.05%. Other surfactants of interest include poloxamer block polymers of polyethylene glycol polypropylene glycol such as Lutrol F-68, Lutrol F-127, etc., e.g. at a concentration of from about 5% to about 10%, preferably about 8%.

For samples that are stored frozen, for example at −20° C. or −₈₀° C., suitable buffers are as described above, however the inclusion of surfactants is generally less important to stability, and may be omitted. Glycerol at a concentration of from about 2% to about 10% may be included.

Adenoviral formulations may be more stable at virus concentrations of from about 10¹¹ to about 2×10¹² particles/ml, and may be less stable at higher concentrations, particularly in liquid formulations (Tables 17 and 18).

The viral particles of the present invention may include classic pharmaceutical preparations for use in therapeutic regimens, including their administration to humans. Administration of therapeutic compositions according to the present invention will be via any common route so long as the target tissue is available via that route. This includes oral, nasal, buccal, rectal, vaginal or topical. Alternatively, administration will be by orthotopic, intradermal subcutaneous, intramuscular, intraperitoneal, or intravenous injection. Such compositions would normally be administered as pharmaceutically acceptable compositions that include physiologically acceptable carriers, buffers or other excipients. For application against tumors; direct intratumoral injection, inject of a resected tumor bed, regional (i.e., lymphatic) or general administration is contemplated. It also may be desired to perform continuous perfusion over hours or days via a catheter to a disease site, e.g., a tumor or tumor site.

An effective amount of the adenovirus vector may be administered to a patient as a composition in a pharmaceutically acceptable excipient (and may or may not be in the same compositions), including, but not limited to, saline solutions, suitable buffers, preservatives, stabilizers, and may be administered in conjunction with suitable agents such as antiemetics. An effective amount is an amount sufficient to effect beneficial or desired results, including clinical results. An effective amount can be administered in one or more administrations. For purposes of this invention, an effective amount of an adenoviral vector is an amount that is sufficient to palliate, ameliorate, stabilize, reverse, slow or delay the progression of the disease state. Some individuals are refractory to these treatments, and it is understood that the methods encompass administration to these individuals. The amount to be given will be determined by the condition of the individual, the extent of disease, the route of administration, how many doses will be administered, and the desired objective.

An effective amount of the therapeutic agent is determined based on the intended goal, for example (i) inhibition of tumor cell proliferation, (ii) elimination or killing of tumor cells, (iii) vaccination, and the like. The term “unit dose” refers to physically discrete units suitable for use in a subject, each unit containing a predetermined-quantity of the therapeutic composition calculated to produce the desired responses, discussed above, in association with its administration, i.e., the appropriate route and treatment regimen. The quantity to be administered, both according to number of treatments and unit dose, depends on the subject to be treated, the state of the subject and the result desired.

Assessment of the efficacy of a particular treatment regimen may be determined by any of the techniques known in the art, including diagnostic methods such as imaging techniques, analysis of serum tumor markers, biopsy, and/or an evaluation of the presence, absence or amelioration of tumor associated symptoms. It will be understood that a given treatment regime may be modified, as appropriate, to maximize efficacy.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### EXPERIMENTAL

### Example 1

### Cell Harvest & Lysis:

Detergent Lysis. Small scale experiments demonstrated that Triton X-100 lysis resulted in increased virus yield compared to control samples lysed either using microfluidization or 3× freeze thaw. The increase in yield was found both when Triton X-100 was added and allowed to lyse the cells, or if the cells were mixed with Triton X-100, frozen and thawed prior to processing.

In a large scale system with a cell cube, addition of Triton X-100 to the system not only lysed the cells, but also removed any plate bound cells from the system. This was clear on visual inspection of the cell cube module with either Triton X-100 treated or citrated saline treated cubes. The Triton X-100 treated cell cube appeared clear, and the parallel plates in the module can be seen by the naked eye. The citrated saline treated module appeared opaque in comparison.

At the time of harvest, 4L of Tris buffer (about 10% of the total volume of the system) containing 10% Triton X-100, 10% glycerol was added to the cell cube system, such the final pH was 8.0. This mixture was continuously re-circulated in the system at 37° C. for 1 hr. This was sufficient to lyse the cells and remove additional adherent cells from the system. The lysed cells were then harvested and processed further. The addition of Triton X-100 for cell lysis and harvest improved virus recovery 5-10 fold.

Harvest with citrated saline & lysis by microfluidization. At the time of harvest, the medium was drained off (and stored), and the cell cube module was filled with 25% of its volume of citrated saline and incubated for 5 hours with occasional tapping with a rubber mallet. The dislodged cells were then drained off and collected, then homogenized using a microfluidizer with 1000 psi until the cells were completely homogenized.

### Example 2

### Clarification & Adjustment of Conductivity Prior to Loading Pall Mustang-Q Columns

The cell harvest was allowed to settle and clarified to 0.2 μm starting with a 0.8 μm Sartorius filter followed by a 0.45 μm filter and by a 0.2 μm filter. The homogenized lysate typically required about 3-4 filters, while the Triton X-100 lysed cells required only 2 filters. Large chunks of cellular debris were seen with the Triton X-100 lysed material, which were allowed to settle before filtration, thus making clarification much simpler. The conductivity of the clarified cell lysate was then increased to about 40 mS/cm with 1M NaCl before purification. This ensured that all the cellular and FBS proteins, Triton X-100 and other contaminants did not bind to the filter, thus making the filter available for adenovirus binding.

### Example 3

### Purification of the Adenovirus

Q-Sepharose purification: The clarified cell lysate was purified on a Q-Sepharose column, by loading at 40 mS/cm, washing the column and then eluting the adenovirus with 50 mS/cm (10 mM Tris, 1 mM MgCl₂, 500 mM NaCl). The remaining bound DNA was eluted with 1.5 mM NaCl. The initial peak represented mostly proteins from 293 cells and fetal bovine serum. The second peak was the virus peak and the last peak was the DNA peak. For a large scale manufacturing system, typically a large column is packed (12 L) and after each use is soaked in 1M NaOH for 2-4 h, and stored in 20% ethanol. However, even with extensive soaking in NaOH, it was impossible to remove all the bound DNA from the column. The typical recovery on this column is between 60-80%. In contrast, when disposable Pall Mustang-Q capsules were used, bound DNA was removed from the column and the yield was substantially higher.

Pall Mustang-Q Filter purification: The clarified cell lysate was purified on Pall Mustang-Q capsules. These disposable filter cartridges are made by coating pleated, polyester sulfone membranes with an anion exchange support. They have a high capacity for both adenovirus (approximately 5×10¹² virus particles/ml) and cellular DNA (25 mg/ml). They can be run with very high flow rates (i.e. 10 cartridge volumes/min vs. 0.1-0.3 column volumes/min for conventional resins), making the process more efficient.

The binding capacity of Pall Mustang-Q filter cartridges was determined by overloading the cartridges and assessing the amount of virus bound. A 0.35 ml Pall Mustang Q filter cartridge (coin filter) was used and 4×10¹² viral particles were loaded on the filter. The Pall Mustang Q filter cartridge was washed with 380 mM NaCl and eluted with 500 mM NaCl and the amount of adenovirus in each fraction was determined by Q-sepharose. Using this method, the amount of bound virus was 2.1×10¹² for the 0.35 ml filter, which represents a binding capacity of 6×10¹² per mL.

Several NaCl concentrations, ranging from 500-600 mM NaCl, were tested for virus elution. No virus peak was evident at 500 mM, while at 525 and 575 mM NaCl, a virus peak was seen with a small trailing peak. At 575 and 600 mM NaCl, a single virus peak was seen, and the virus yield was the best at 600 mM NaCl. Based on these results, 600 mM was the optimal elution concentration for NaCl. The trailing peak may represent a minor heterogeneity in the virus.

In order to determine the potential of the cartridge for re-use, five consecutive runs were carried out on a Pall Mustang Q filter cartridge. The sample was loaded, washed with 380 mM NaCl, with the virus eluted at 500 mM NaCl and the DNA eluted with 2000 mM NaCl. The process was repeated for 5 runs, at which point a decrease in the virus peak was seen. After the fifth run, the cartridge was washed with 0.5 M NaOH and the virus was re-run on the cartridge. The virus recovery was maintained for 4 runs, dropped in the fifth run and was regained in the sixth run after the NaOH wash. The amount of virus was monitored on Q-Sepharose. The following table represents the virus recovery after each run.

TABLE 1

| Run # | Virus Recovery (%) |
|---|---|
| 1 | 100 |
| 2 | 117 |
| 3 | 119 |
| 4 | 108 |
| 5 | 88 |
| 0.5M NaOH wash | |
| 6 | 116 |

### Example 4

### Removal of Residual DNA with Benzonase

Although the majority of the DNA contaminants are removed following purification with Pall Mustang Q filters, there still remains detectable levels of DNA (about 5-15 ng/1×10¹² viral particles). There are several ways to reduce this DNA, one of which is treatment with Benzonase, a nuclease that digests the DNA. The added Benzonase needs to be removed from the final process, and anion exchange chromatography effectively removes the Benzonase. The Benzonase can be added either directly to the cell harvest or after the initial purification step, which reduces the amount of Benzonase needed. However, this also implies that an additional purification is needed to remove the Benzonase. This is achieved by using another Pall Mustang-Q filter cartridge. The use of the cartridge is preferable over the use of Q-Sepharose column, beacuse of improved recovery (90% compared to 70%); higher flow rates; and higher binding capacity. This additional chromatography step also ensures that any residual Triton X-100, proteins or DNA is removed.

The following example demonstrates the amount of Benzonase needed to digest the DNA in the virus sample. In this example, the virus was treated with various amounts of Benzonase for either 2 hr or 20 hr and the sample run on an analytical Q-Sepharose column. The areas underneath the virus and the DNA peaks were determined to obtain a quantitative measure of the virus and DNA. The results are tabulated below, where virus A260(e6) and DNA A260 (e6) represents the areas under the two peaks.

TABLE 2

| | Virus | | | |
|---|---|---|---|---|
| | A260 10⁶) | 24 hr | DNA A260 (10⁶) | 24 hr |
| Units/mL | 2 hr incubation | incubation | 2 hr incubation | incubation |
| Control (0) | — | 21.6 | 27.7 | 26.2 |
| 50 | 22.1 | — | 9.1 | — |
| 100 | — | 23.6 | 9.3 | 9.3 |
| 250 | 23.6 | 21.3 | 7.4 | 7.4 |

According to these data, Benzonase degrades the majority of the DNA within 2 hours. None of the samples showed a decrease in the DNA peak after this point. There does not seem to be any advantage with increasing the concentration beyond 50 U/mL.

### Example 5

### Purification of Adenovirus Using 2-Step Purification Procedure

A scheme for the harvest and purification of adenovirus utilized the following steps: cell harvest and lysis using 1% Triton X-100, clarification with 3 filters: 0.8 μM, 0.8 μM, 0.2 μM, purification on a Pall Mustang-Q filter cartridge, treatment with Benzonase, followed by another purification on a Pall Mustang-Q filter cartridge, concentration/diafiltration and sterile filtration.

This procedure was first tested using small Mustang-Q Pall cartridge filters, with a fresh filter for the second step.

The cell lysate was clarified, adjusted for NaCl concentration and five 15 mL runs were performed on the coin filter (15 mL gave a loading ratio of 5×10¹² particles/mL of filter). The eluates were collected and pooled then diluted 50% with Dl water (diluted pool=65 mL) and incubated with 50 units/mL Benzonase at 4° C. overnight. The next day, two loads were purified over the same filter as used in the first Pall step and two loads over a new (virgin) filter. The results are provided below:

TABLE 3

| | | Vol- | | Re- | |
|---|---|---|---|---|---|
| | Particles/ | ume | Total | covery | Purity |
| Sample | (mL) | (mL) | Particles | (%) | (%) |
| Pall 1 load | | | 10 × 10¹³ | | |
| Pall 1 eluates | 1.5 × 10¹¹ | 65 | 9.8 × 10¹² | 98 | 71 |
| (dil) | | | | | |
| Pall 2 eluate | 7.1 × 10¹¹ | 4 | 2.8 × 10¹² | See | 98 |
| (used filter) | | | | below | |
| Pall 2 eluate | 7.3 × 10¹¹ | 4 | 2.9 × 10¹² | 79 | 99 |
| (new filter) | | | | | |

Concentration and diafiltration was performed using a hollow-fiber concentrator with 500 kD MWCO from A/G technology.

Sterile filtration is the final step in this process and historically has caused a 20-30% loss during filtration. In order to minimize the loss at this step, Pall and Sartorius filters were compared to the Millipak filters currently used in manufacturing. The viral load was normalized to the surface area of the filters. Two modifications were made in the process. There was an initial step with buffer to wet the surface of the filters and then after filtration, a rinse step was included to recover more virus. Several aliquots were collected with a view to determine the step at which maximum loss occurred.

The initial experiment showed that a Millipak filter resulted in the best virus recovery and a Sartorius filter gave the worst performance. Initial results also showed that most virus loss occurs in the first aliquot collected, suggesting that the virus is non-specifically absorbed by the filter and once the surface is saturated, virus loss is minimized.

TABLE 4

| | Surface | Volume | | | |
|---|---|---|---|---|---|
| Filter | Area | Filtered | Original (vp) | Final (vp) | Yield (%) |
| Millipak-20 | 100 | 150 | 3.00 × 10¹⁴ | 2.70 × 10¹⁴ | 90 |
| Pall | 500 | 400 | 8.00 × 10¹⁴ | 5.60 × 10¹⁴ | 70 |
| Pall | 500 | 250 | 2.43 × 10¹⁴ | 1.70 × 10¹⁴ | 70 |
| Sartorius | 150 | 250 | 2.50 × 10¹⁴ | 1.04 × 10¹⁴ | 42 |

This experiment was then repeated using a Millipak 40 (surface area 200 cm²) with, a load of 2×10¹⁵ viral particles in 800 ml. The filter was wetted with 100 ml of the buffer and all the buffer was pushed through prior to the introduction of the virus. 100 ml aliquots were collected. The final loss using this method was 4%. This experiment also shows that if only 0.46×10¹⁵ viral particles were loaded, the loss was much higher, at 11%. Therefore, the procedure uses the same Millipak filters, but incorporates an initial washing step, and loads of at least 1×10¹³ viral particles/cm².

TABLE 5

| | | | | | total particles | total load in | |
|---|---|---|---|---|---|---|---|
| | load vol | | total particles | step | in pooled | pooled | |
| | (ml) | vp/ml | in fraction | recovery | fractions | fractions | % recovery |
| 1 | 100 | 1.94 × 10¹² | 1.94 × 10¹⁴ | 84% | | | |
| 2 | 100 | 2.18 × 10¹² | 2.18 × 10¹⁴ | 94% | 4.12 × 10¹⁴ | 4.64 × 10¹⁴ | 89% |
| 3 | 100 | 2.21 × 10¹² | 2.21 × 10¹⁴ | 95% | | | |
| 4 | 100 | 2.24 × 10¹² | 2.24 × 10¹⁴ | 97% | 8.57 × 10¹⁴ | 9.28 × 10¹⁴ | 92% |
| 5 | 100 | 2.23 × 10¹² | 2.23 × 10¹⁴ | 96% | | | |
| 6 | 100 | 2.27 × 10¹² | 2.27 × 10¹⁴ | 98% | 1.31 × 10¹⁵ | 1.392 × 10¹⁵ | 94% |
| 7 | 100 | 2.24 × 10¹² | 2.24 × 10¹⁴ | 97% | | | |
| 8 | 100 | 2.28 × 10¹² | 2.28 × 10¹⁴ | 98% | 1.76 × 10¹⁵ | 1.856 × 10¹⁵ | 95% |
| 9 | 50 (wash) | 5.00 × 10¹¹ | 2.50 × 10¹³ | | 1.78 × 10¹⁵ | 1.856 × 10¹⁵ | 96% |

### Example 6

### Large-Scale Purification of Adenovirus

The process described in Example 5 was tried out on a large scale. In this method, the same Pall Mustang filter cartridge was used twice, for both purification steps. The homogenized, clarified, cell lysate was first purified on a Pall Mustang filter cartridge followed by Benzonase treatment and re-purified on the same filter cartridge. The yields and recoveries after each step are outlined below:

TABLE 6

| | Volume | | | Total | | Particle/ | Step | Overall |
|---|---|---|---|---|---|---|---|---|
| Process Step | (mL) | # vp/ml | pfu/ml | Particles | Total PFU | PFU | recov. | recov. |
| Harvest w/Triton | 31000 | 1.95 × 10¹¹ | 1.28 × 10¹⁰ | 6.0 × 10¹⁵ | 3.97 × 10¹⁵ | 15 | | |
| Clarification | 31000 | 1.90 × 10¹¹ | 9.35 × 10⁹ | 5.89 × 10¹⁵ | 2.90 × 10¹⁴ | 20 | 97% | 97% |
| Purification | 7800 | 6.41 × 10¹¹ | 4.23 × 10⁹ | 5.00 × 10¹⁵ | 3.30 × 10¹³ | 152* | 85% | 83% |
| Benzonase | 7800 | 6.41 × 10¹¹ | 5.13 × 10⁹ | 5.00 × 10¹⁵ | 4.00 × 10¹³ | 125* | 100% | 83% |
| treatment | | | | | | | | |
| Re-purification | 3450 | 1.42 × 10¹¹ | 2.50 × 10¹⁰ | 4.90 × 10¹⁵ | 8.63 × 10¹³ | 57* | 98% | 81% |
| Conc., | 3450 | 1.42 × 10¹¹ | 9.00 × 10¹⁰ | 4.90 × 10¹⁵ | 3.11 × 10¹⁴ | 16 | 100% | 81% |
| Diafiltration | | | | | | | | |
| Sterile Filtration | | | | | | | 95% | 77% |

The initial and the final Particle/PFU ratio are the same, however those samples marked with an asterisk were frozen without glycerol, and the high particle/PFU ratio may be attributed to that freezing.

The overall recovery was 77% after final sterile filtration, which is a great improvement over the prior process. This process also requires less time to complete, less buffer for purification, and a highly concentrated eluate (requiring less concentration time).

Residual DNA in the samples was measured at each point and these values are provided below. After the first Pall filter cartridge step, the DNA was undetectable in the assay. However, after the second Pall filter cartridge step, there was an increase in the DNA content, which may be attributable to the use of the same cartridge for both runs, suggesting that performance may benefit from using a virgin filter cartridge for each step.

TABLE 7

| Sample | DNA Copy # | pg DNA/10⁹ virus |
|---|---|---|
| Pall 1 eluate | 95 | 474 |
| Diluted Pall 1 eluate −Benz | 269 | 1345 |
| Diluted Pall 1 eluate +Benz | 0 | 0 |
| Pall 2 eluate (from P1 −Benz) | 24 | 120 |
| Pall 2 eluate (from P1 +Benz) | 26 | 130 |
| Diafiltered Pall 2 eluate | 25 | 125 |
| (from P1 +Benz) | | |

A comparison was performed against the previously used process, where lysis was performed with citrated saline and homogenization, and the virus was then purified on large scale Q-Sepharose. The following table provides an example from one run. The overall recovery in this run was 29% relative to 77% in the process described above.

TABLE 8

| | Volume | | | Step | Overall |
|---|---|---|---|---|---|
| Process Step | (mL) | # vp/ml | Total Particles | recovery | recovery |
| Harvest | 47,300 | 3.2 × 10¹⁰ | 1.5 × 10¹⁵ | | |
| Homogenization | 47,600 | 2.8 × 10¹⁰ | 1.3 × 10¹⁵ | 87% | |
| Clarification | 51500 | 2.90 × 10¹¹ | 1.5 × 10¹⁵ | | 99% |
| Purification | 3500 | 1.5 × 10¹¹ | 5.00 × 10¹⁴ | 35% | 35% |
| Conc., Diafiltration | 3450 | 1.42 × 10¹² | 4.90 × 10¹⁴ | 96% | 32% |
| Sterile Filtration | | | 4.43 × 10¹⁴ | 91% | 29% |

This run had an especially poor recovery in the Q-Sepharose step. The following table compares the particle numbers and recoveries after the purification step for the Q-sepharose column vs. Triton/Pall filter cartridge methods.

TABLE 9

| Process | N | # viral particles |
|---|---|---|
| Q-sepharose | 7 | 1.3 × 10¹⁵ + 0.7 × 10¹⁵ |
| Triton/Pall filter | 2 | 8.7 × 10¹⁵ + 0.4 × 10¹⁵ |

It can be seen here that the process of the present invention yielded 6.7-fold more virus than the older Q-sepharose process, a very significant improvement, particularly for large scale preparations such as those required for clinical trials.

### Example 7

### Formulations

The effects of various additives to formulations of virus were tested. Lyophilized virus may show signs of aggregation upon storage for lengthy periods of time. In addition, lyophilization can be a long, complex and expensive process. Frozen formulations have shown extended stability at −80° C., however, once thawed, the virus may precipitate under certain conditions. Due to these considerations, a series of investigations were set up to assess the stability of adenovirus at 5° C. and −20° C. Variables included the use of Tris buffer instead of PBS, and the inclusion of surfactants and preservatives. ARCA lyophilization buffer (5% sucrose, 1% glycine, 1 mM MgCl₂ and 10 mM Tris) was used to study the effects of added surfactants e.g. Lutrol F-127, Lutro F-68, PEG 3350.

The tested formulations include the following:

TABLE 10

| | | | | | | Storage |
|---|---|---|---|---|---|---|
| | Date | Virus conc. | Virus | Buffer | Storage condition | temp |
| B series | Aug. 22, 2000 | 5 × 10¹¹ | CV890 | ARCA | Under N₂ | 5, 25 |
| C series | Oct. 11, 2000 | 2 × 10¹² | CV706 | Tris | Under N₂ | 5, 25 |
| D series | Oct. 11, 2000 | 2 × 10¹² | CV787 | Tris | Under N₂ | 5, 25 |
| V series | Oct. 20, 2000 | 5 × 10¹¹ | CV890 | Tris | Under room air | 5, 35 |
| H series | Jan. 08, 2001 | 3.5 × 10¹² | CV706 | Tris | Under N₂ | 5, 25, −20 |
| G series | Apr. 02, 2001 | 2.0 × 10¹² | CV787 | Tris | Under room air | 5, 25, −20 |

Results:

B Series:

A total of 11 formulations was set up with CV890 in ARCA buffer and sodium citrate and filled in glass vials under N₂. The formulations in sodium citrate were very unstable and were dropped off the study. The remaining formulations are tabulated below.

TABLE 11

| | 1B | 2B | 3B | 4B | 10B | 11B (control) |
|---|---|---|---|---|---|---|
| Sucrose | 5% | 5% | 5% | 5% | 5% | |
| Glycine | 1% | 1% | 1% | 1% | 1% | |
| MgCl₂ | 1 mM | 1 mM | 1 mM | 1 mM | 1 mM | |
| Tris | 10 mM | 10 mM | 10 mM | 10 mM | 10 mM | PBS |
| Surfactant | Tween 80, | F-127, 8% | F-68, 8% | PEG, 5% | F-127, 8% | 10% Glycerol |
| | .05% | | | | | |
| Preservative | None | None | None | None | Benyl Alc. | |
| Temp | 5,25 | 5, 15, 25, 35 | 5, 25 | 5, 25 | 5, 25 | −80, 5 |

Formulation 2B was used in a stress study, which is discussed below.

The samples were visually inspected and tested periodically in biological assay (plaque-forming assay) for infectivity. Viral particle counts were determined by UV and HPLC. The results are as follows:

TABLE 12
Plaque forming Units (PFU) at 5 deg C.

| | 0 | 1 mo | 2 mo | 4 mo | 6 mo |
|---|---|---|---|---|---|
| 1B | 5.0E+10 | 3.6E+10 | | 4.9E+10 | 6.5E+10 |
| (5° C.) | | | | | |
| 2B | 4.1E+10 | 3.2E+10 | | 4.9E+10 | 1.1E+11 |
| (5° C.) | | | | | |
| 3B | 3.7E+10 | 3.2E+10 | 3.7E+10 | 4.4E+10 | 4.5E+10 |
| (5° C.) | | | | | |
| 4B | 4.1E+10 | 3.2E+10 | 4.9E+10 | 5.1E+10 | 5.2E+10 |
| (5° C.) | | | | | |
| 10B | 5.2E+10 | 3.4E+10 | 2.7E+10 | 3.6E+10 | 4.5E+10 |
| (5° C.) | | | | | |
| 11B | 6.1E+10 | 3.6E+10 | | 5.1E+10 | 6.2E+10 |
| (−80°) | | | | | |
| 11B | 6.1E+10 | 3.8E+10 | | 4.1E+10 | 3.4E+10 |
| (5° C.) | | | | | |

TABLE 13
particle number by HPLC at 5 deg C.

| | 0 | 1 mo | 2 mo | 4 mo | 6 mo |
|---|---|---|---|---|---|
| 1B (5° C.) | 5.5E+11 | 5.7E+11 | 4.8E+11 | 4.4E+11 | 4.6E+11 |
| 2B (5° C.) | 2.9E+11 | 2.3E+11 | 2.5E+11 | 3.6E+11 | 1.8E+11 |
| 3B (5° C.) | 4.6E+11 | 5.1E+11 | 3.1E+11 | 3.6E+11 | 2.6E+11 |
| 4B (5° C.) | 5.3E+11 | 5.3E+11 | 4.7E+11 | 4.4E+11 | 4.4E+11 |
| 10B (5° C.) | 4.7E+11 | 2.0E+11 | 1.2E+11 | 3.5E+11 | 1.8E+11 |
| 11B (5° C.) | 3.5E+11 | 3.2E+11 | 5.2E+11 | 4.8E+11 | 4.0E+11 |

TABLE 14
Plaque forming Units (PFU) at 25 deg C.

| | 0 | 1 mo | 2 mo | 4 mo | 6 mo |
|---|---|---|---|---|---|
| 1B (25° C.) | 5.0E+10 | 1.8E+10 | 3.1E+10 | 2.7E+10 | 1.2E+10 |
| 2B (25° C.) | 4.1E+10 | | 2.7E+10 | 3.3E+10 | 8.0E+09 |
| 3B (25° C.) | 3.7E+10 | | 2.4E+10 | 1.1E+10 | 5.0E+08 |
| 4B (25° C.) | 4.1E+10 | | 3.6E+09 | | |
| 10B (25° C.) | 5.2E+10 | | 2.1E+10 | | |
| 11B (25° C.) | 61E+10 | | 2.8E+10 | 1.4E+10 | 2.3E+09 |

TABLE 15
Particle number by HPLC at 25 deg C.

| | 0 | 1 mo | 2 mo | 4 mo | 6 mo |
|---|---|---|---|---|---|
| 1B | 5.5E+11 | 5.5E+11 | 5.0E+11 | 4.8E+11 | 4.7E+11 |
| (25° C.) | | | | | |
| 2B | 2.9E+11 | 2.2E+11 | 1.7E+11 | 3.2E−11 | 2.1E+11 |
| (25° C.) | | | | | |
| 3B | 4.6E+11 | 2.7E+11 | 1.4E+11 | 2.6E+11 | 2.9E+10 |
| (25° C.) | | | | | |
| 4B | 5.3E+11 | 5.1E+11 | 6.2E+10 | 4.8E+10 | |
| (25° C.) | | | | | |
| 10B | 4.7E+11 | 4.7E+11 | 1.9E+11 | 2.6E+11 | 1.9E+11 |
| (25° C.) | | | | | |
| 11B | 3.5E+11 | 6.3E+11 | 4.9E+11 | 4.5E+11 | 3.8E+11 |
| (25° C.) | | | | | |

At 5° C., 1B, 2B, 4B and 11B exhibited the best stability. However, at 25° C., virus stored in 4B showed a decrease of 1 log of infectivity at 2 months and had no activity at 6 months and virus stored in 11B had a 2 log decrease in activity during the same time period. Virus stored in 1B and 2B exhibited a much smaller decrease in activity after 6 months of storage at 25° C.

Based on these observations, formulations 1B and 2B appear to be the best, retaining real time stability at 5° C. for 6 months and showing the lowest decrease in activity after storage at 25° C. for 6 months. 1B is ARCA buffer, 0.05% Tween. 2B is ARCA buffer, 8% Lutrol F-127 (Poloxamer 407 (DAC, USP-NF); a block polymer consisting of 73% of polyethylene glycol and 27% polypropylene glycol with an average molecular weight of 12,000). The results with PEG 3340 (4B) indicate excellent stability at 5° C., but a lack of stability at 25° C.

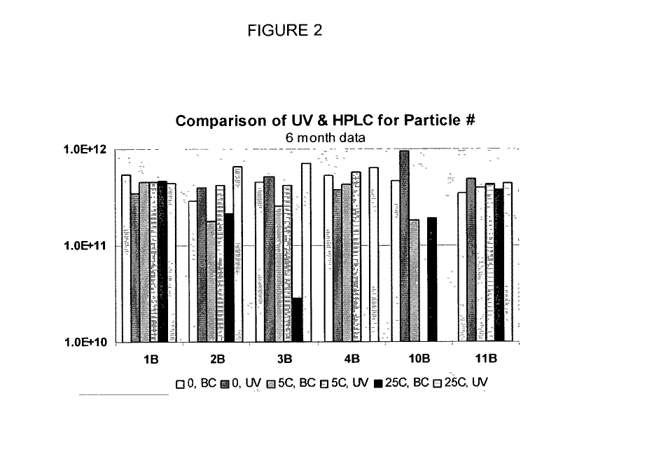

Particle counts were measured based on both UV and HPLC at the 6-month time point. While the spectrophotometric method measures all species that absorb at 260 nm and 280 nm, and thus does not distinguish between the integral viral particle and the dissociated proteins and DNA, the HPLC method measures the integral viral particles. However, the HPLC method does not distinguish intact, active virus particles from virus particles that have parts of the capsid proteins denatured or inactive. Moreover, it does not separate the empty capsid very well from the intact virus. FIG. 2 shows the comparison between the two methods:

The first two bars in this figure represent the zero time values, while the rest represent the 6-month value. Bars denoted by UV are the particle counts as measured by UV and those with BC are particle counts measured by the BioCAD (HPLC). Adenovirus particle number in formulation 1B at either 5° or 25° C. remains unchanged after 6 months, whether UV or HPLC is used for the particle number determination. Formulation 2B at 25° C. exhibits a slight decrease in particle numbers by HPLC, which represents the integral viral particle. The losses in activity in 3B and 4B at 25° C. are reflected in the decrease in viral particle number seen by HPLC. The particle number by UV remains unchanged. This shows that although the virus may have dissociated, the number of absorbing species have not changed. Based on these analyses, 1B appears to be the best formulation.

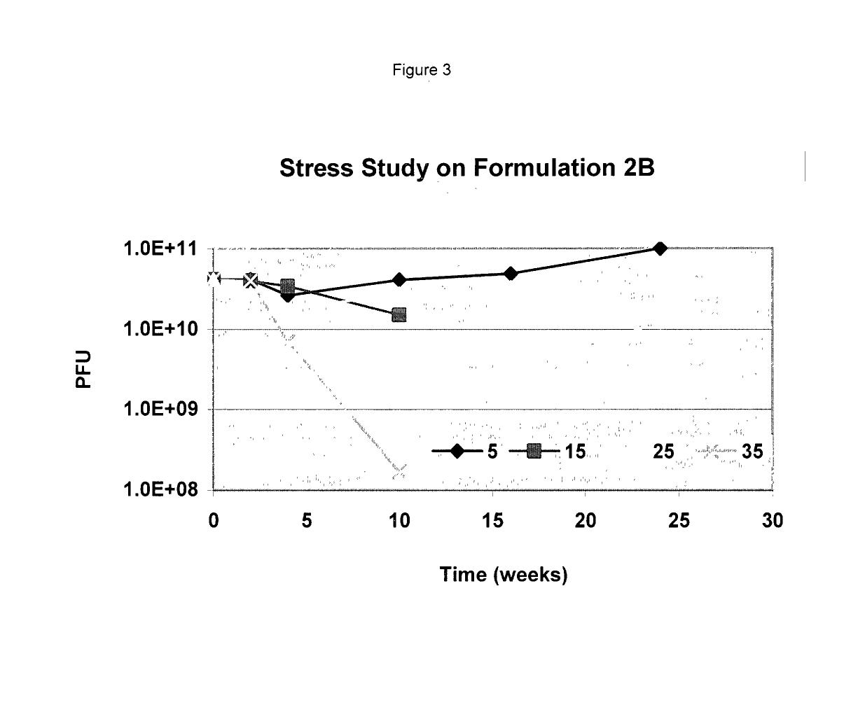

A stress study was done on formulation 2B, by assessing CV890 stability at 5, 15, 25 & 35° C. Plaque assay and particle counts were performed every 2 weeks for 10 weeks. The results are shown in FIG. 3. These data show that there is a 2-3 log reduction in activity when CV890 is stored at 35° C. for 10 weeks, while there is no appreciable change at either 5, 15 or 25° C. After 6 months of storage however, there is 0.5-1 log reduction in activity at 25° C.

The two formulations that show very good liquid stability at 5° C. are formulations 1B and 2B. All these studies were carried out at a particle concentration of 5×10¹¹ per ml. Adenoviral storage at −20° C. is another alternative. A series of formulations were set up in the frozen and liquid state. Three viruses: CV706, CV787 and CV890, were tested with a variety of formulations at a viral concentration for from 0.5 to 1.0×10¹² particles/ml. The stability profile, for most formulations looks similar to that of the 1B & 2B formulation. The results of the two studies were compared at 350, and the observed stability profiles were very similar. It was also found that storing vials under N₂ may not be necessary for short-term stability of the adenovirus. No loss of titer was observed after 21 months of storage at 5° C. using two liquid formulations, VA1 (2 mM MgCl₂, 10 mM Tris, 1% Glycine, 3% Sucrose, 10% Glycerol, pH=7.8) and VC1 (1.6 mM MgCl₂, 8 mM Tris, 0.8% Glycine, 2.4% Sucrose, 8% Glycerol, Lutrol, 3.4%, pH=7.8), when tested with the CV890 virus at 0.5×10¹² vp/ml.

### Example 8

### Stability of Formulations at High Particle Concentration

Further experiments were set up to study adenoviral stability at high particle concentrations using CV706. Tris was the buffer of choice. The formulations are shown in Table 16.

TABLE 16

| | 1H | 2H | 2H | 3H | 4H | 5H | 5H lo |
|---|---|---|---|---|---|---|---|
| Sucrose | | 5% | 5% | | 2% | 6% | 6% |
| Glycine | | 1% | 1% | | 1% | 1% | 1% |
| MgCl₂ | 1 mM | 1 mM | 1 mM | 1 mM | 1 mM | 1 mM | 1 mM |
| Tris | PBS | 10 mM | 10 mM | 10 mM | 10 mM | 10 mM | 10 mM |
| Glycerol | 10% | | | 10% | 5% | 2% | 2% |
| Surfactant | None | Tween, | Tween, | None | None | None | None |
| | | 0.05% | 0.05% | | | | |
| Preservative | None | None | BHA | None | None | None | None |
| Temp | −20, 5, 25 | −20, 5, 25 | −20, 5, 25 | −20, 5, 25 | −20, 5, 25 | −20, 5, 25 | −20, 5, 25 |
| pH | 7.6 | 7.6 | 7.6 | 7.7 | 7.7 | 7.7 | 7.7 |
| Viral part. # | 3.8 × 10¹² | 3.4 × 10¹² | 3.4 × 10¹² | 3.4 × 10¹² | 3.1 × 10¹² | 3.4 × 10¹² | 2.2 × 10¹² |

TABLE 17
Stability of CV706 at 5 deg C.

| | 0 | 30 | 90 |
|---|---|---|---|
| 2H | 3.40E+11 | 1.90E+11 | 0 |
| 2H BHA | 5.30E+11 | 2.00E+11 | 0 |
| 3H | 2.50E+11 | 2.60E+11 | 8.50E+10 |
| 4H | 2.40E+11 | 1.60E+11 | 4.10E+10 |
| 5H | 9.60E+11 | 2.40E+11 | 8.40E+10 |
| 5H lo | 1.50E+11 | 1.60E+11 | 1.20E+11 |

TABLE 18
Stability of CV706 at −20 deg C.

| | 0 | 30 | 90 |
|---|---|---|---|
| 2H | 2.80E+11 | 3.50E+11 | 1.50E+11 |
| 2H BHA | 3.40E+11 | 1.90E+11 | 1.30E+11 |
| 3H | 2.50E+11 | 3.90E+11 | 1.30E+11 |
| 4H | 2.40E+11 | 2.40E+11 | 1.20E+11 |
| 5H | 9.60E+11 | 2.70E+11 | 1.40E+11 |
| 5H lo | 1.50E+11 | 1.60E+11 | 1.20E+11 |

These results demonstrate the difficulty of storage at very high viral concentrations. For example, the results shown for 5H and 5H lo show the drop in titer of high concentration formulations compared to lower concentrations. While the infectivity of CV706 in 5H decreased after 2 months, 5H lo retains its infectivity. The results for 5H and 5H lo indicate that the ARCA buffer (5% sucrose, 1% glycine, 1 mM MgCl₂ and 10 mM Tris) is an excellent buffer for storage of 2.2×10¹² viral particles per ml, but is less stable at a concentration of 3.4×10¹² viral particles per ml.

What is claimed is:

1. A method for producing substantially pure replication competent adenovirus, the method comprising:
lysing cells infected with said replication competent adenovirus with a non-ionic detergent;
clarifying the lysate of said adenovirus infected cells by passing over a depth filter;
binding said adenovirus to a first anionic exchange filter, wherein said first anionic exchange filter is a high throughput filter cartridge;
eluting said adenovirus from said first anionic exchange filter at an ionicity that permits separation of the adenovirus from major cellular contaminants;
adding nuclease to the adenovirus containing eluate to substantially digest free nucleic acids present in said eluate;
binding said adenovirus to a second anionic exchange filter;
eluting said adenovirus from said second anionic exchange filter at an ionicity that permits separation of the adenovirus from major cellular contaminants;
wherein said eluant comprises substantially pure replication competent adenovirus.

2. The method according to claim 1, wherein said non-ionic detergent is Triton X-100 or NP-40.

3. The method according to claim 2, wherein said non-ionic detergent is added a concentration of at least about 0.5% and not more than about 2.5%.

4. The method according to claim 3, wherein said non-ionic detergent is in contact with said infected cells for at least about 30 minutes and not more than about 4 hours.

5. The method according to claim 1, wherein said nuclease is benzonase.

6. The method according to claim 1, wherein said anionic exchange filter comprises quaternary ammines as the anion exchanger.

7. The method according to claim 6, wherein said anionic exchange filter is a Pall Mustang Q filter cartridge.

8. The method according to claim 1, wherein said second anionic exchange filter is a high throughput filter cartridge.

9. The method according to claim 1, wherein said first anionic exchange filter and said second anionic exchange filter are the same.

10. The method according to claim 1, wherein said first anionic exchange filter and said second anionic exchange filter are different.

11. The method according to claim 10 wherein said cells infected with said replication competent adenovirus is grown in suspension.

12. The method according to claim 11, wherein the yield of adenovirus is at least about 80% of the adenovirus present in said lysate of said adenovirus infected cells.

13. The method according to claim 11, wherein the yield of adenovirus is at least about 85% of the adenovirus present in said lysate of said adenovirus infected cells.

14. The method according to claim 11, wherein the yield of adenovirus is at least about 90% of the adenovirus present in said lysate of said adenovirus infected cells.

15. An improved formulation for storage of infection competent adenovirus, said improvement comprising a liquid formulation comprising glycine at a concentration of at least about 0.5% and not more than about 1.5%.

16. The formulation according to claim 15, wherein said formulation further comprises a non-ionic detergent at a concentration of from about 0.01% to about 0.1%.

17. The formulation according to claim 15, wherein said formulation further comprises a poloxamer block polymer at a concentration of from about 5% to about 10%.

18. The formulation according to claim 15, wherein said formulation provides enhanced stability of infection competent adenovirus at 5° C.

19. The formulation according to claim 16, wherein said formulation comprises 1% glycine and said non-ionic detergent is Tween 80.

20. The formulation according to claim 17, wherein said formulation comprises 1% glycine and said poloxamer block polymer is F-27.
